(19) United States
(12) Patent Application Publication
(10) Pub. No.: US 2014/0121768 A1
(43) Pub. Date: May 1, 2014

(54) ACCOMMODATING INTRAOCULAR LENS WITH CILIARY BODY ACTIVATION

(71) Applicant: Novartis AG, Basel (CH)

(72) Inventor: MICHAEL J. SIMPSON, ARLINGTON, TX (US)

(73) Assignee: NOVARTIS AG, Basel (CH)

(21) Appl. No.: 14/034,102

(22) Filed: Sep. 23, 2013

Related U.S. Application Data
(60) Provisional application No. 61/720,688, filed on Oct. 31, 2012.

(51) Int. Cl. A61F 2/16

(52) U.S. Cl. CPC A61F 2/1624; A61F 2/1648
USPC 623/6.32; 623/6.37

(57) ABSTRACT

An accommodative lens assembly includes a lens body defining an optic lens, a haptic system, and a wing. The lens body is formed and implanted into an eye in a disaccommodative configuration. The haptic system includes one or more haptics that support the optic lens and transmits forces from an anatomical structure such as a ciliary body of the eye, causing the optic lens to deform into an accommodative configuration. In order to stabilize the optic lens so that the optic lens is not displaced from its implantation site, the wing anchors the optic lens within an anterior capsulorhexis of the capsular bag such that the transmitted forces that deform the optic lens during accommodation do not also displace the optic lens from its implanted position. When implanted, the optic lens is anterior to the capsular bag.

This application claims the priority of U.S. Provisional Patent Application No. 61/720,688 filed on Oct. 31, 2012.

### FIELD OF THE INVENTION

The present disclosure relates to an accommodative lens and in particular to an intraocular lens that accommodates in response to movement by a ciliary body of a patient's eye and is anchored to the capsular bag to prevent displacement of the intraocular lens during accommodation.

### BACKGROUND OF THE INVENTION

A cataract can occur when the natural lens of an eye or its surrounding transparent membrane becomes clouded, resulting in various degrees of blindness. One method of treating this condition is to perform cataract surgery, which involves removing the cataract and implanting an intraocular lens (“IOL”). Some conventional replacement IOLs are rigid and not intended to flex or provide accommodation and therefore requires the patient to use external vision correction such as eyeglasses or contact lenses for near vision. Other conventional IOLs may provide accommodation, but have drawbacks. For instance, movement or displacement of accommodating IOLs typically can create spaces in which material such as cells may accumulate, resulting in posterior capsular opacification, or clouding of the IOL.

It therefore can be seen that a need exists for an accommodating IOL that addresses the foregoing and other related and unrelated problems in the art.

### SUMMARY OF THE INVENTION

According to various implementations of the invention, an accommodative intraocular lens (IOL) is formed having a lens body that is in a disaccommodative configuration (i.e., has a curvature that is in a disaccommodated shape). The lens body is attached, directly or indirectly, to an anatomical structure of an eye such as a ciliary body. In some implementations, the lens body is attached to the ciliary body via a haptic system that includes one or more haptics, which help support and transmit an accommodative force to the lens body. For example, the haptics may transmit an axial compressive force from the ciliary body to the lens body during accommodation of the patient's eye. The transmitted force causes the lens body to alter its shape from a disaccommodative shape to an accommodated shape, thereby changing the power of the IOL. In some implementations of the invention, in order to prevent movement of the lens body while the force is being transmitted, one or more wings may project from and help anchor the lens body, directly or indirectly, within the eye. For example, the wing(s) may anchor the lens body to the capsular bag such that the lens body is not displaced from its implanted location while the transmitted force deforms the lens body.

In some implementations of the invention, the accommodative lens assembly includes a first lens including a lens body, haptic system, and at least one wing projecting from the lens body. The accommodative lens assembly can be implanted in a patient's eye in a position to respond to forces transmitted by the ciliary body during accommodation. In order to stabilize the accommodative lens assembly so that the lens body is not displaced from its implantation site, the wing anchors the accommodative lens assembly in the capsular bag such that the transmitted forces that deform the lens body during accommodation do not also move the lens body from its implanted position. When implanted, the lens body of the accommodative lens assembly generally is anteriorly located with respect to the capsular bag.

In further implementations of the invention, a second intraocular lens can replace the natural lens within the capsular bag. In these implementations, an incision or capsulorhexis is made to remove the natural lens and implant the second intraocular lens. In some implementations, the wing of the first lens is configured to be attached to or otherwise tucked into the capsulorhexis. In some implementations, the second intraocular lens is configured to maintain or otherwise provide a base power during accommodation. In these implementations, the lens body of the first lens can change optical power during accommodation while the second intraocular lens generally does not change optical power. In this manner, the second intraocular lens may substantially maintain its shape while only the lens body of the first lens changes shape during accommodation.

Additionally, in some implementations of the invention, the lens body of the first lens is formed having an inner portion and an outer portion. During accommodation, when the transmitted force deforms the lens body, at least a portion of the inner portion bulges forwardly in a direction away from the capsular bag. In addition, the inner portion can be formed from a thinner, less dense or more flexible membrane material than the outer portion, thereby enabling bulging of the inner portion when force is transmitted to the lens body. In some implementations, the haptic system includes a plurality of haptics each configured to provide a deforming force to the inner portion of the lens body at radial angles during accommodation.

Various objects, features and advantages of the present invention will become apparent to those skilled in the art upon a review of the following Detailed Description of the Invention, when taken in conjunction with the accompanying drawings.

### BRIEF DESCRIPTION OF THE DRAWINGS

The accompanying drawings, which are incorporated into and constitute a part of this specification, illustrate one or more examples of implementations of the invention and, together with the description, serve to explain various principles and aspects of the invention.

FIG. 1 is a side view of an accommodating lens assembly according to the principles of the present invention implanted in a patient's eye.

FIG. 2 is a side view in cross-section of an accommodating lens assembly, according to various implementations of the invention.

FIG. 3 is a plan view of an accommodating lens assembly, according to various implementations of the invention.

FIG. 4 is an exploded view taken in cross-section of an accommodating lens assembly implanted into an eye, according to various implementations of the invention.

FIG. 5 is a plan view of an accommodating lens assembly anterior to a capsular bag, according to various implementations of the invention.

### DETAILED DESCRIPTION OF THE INVENTION

The accompanying drawings, which are incorporated into and constitute a part of this specification, illustrate one or more examples of implementations of the invention and, together with the description, serve to explain various principles and aspects of the invention.

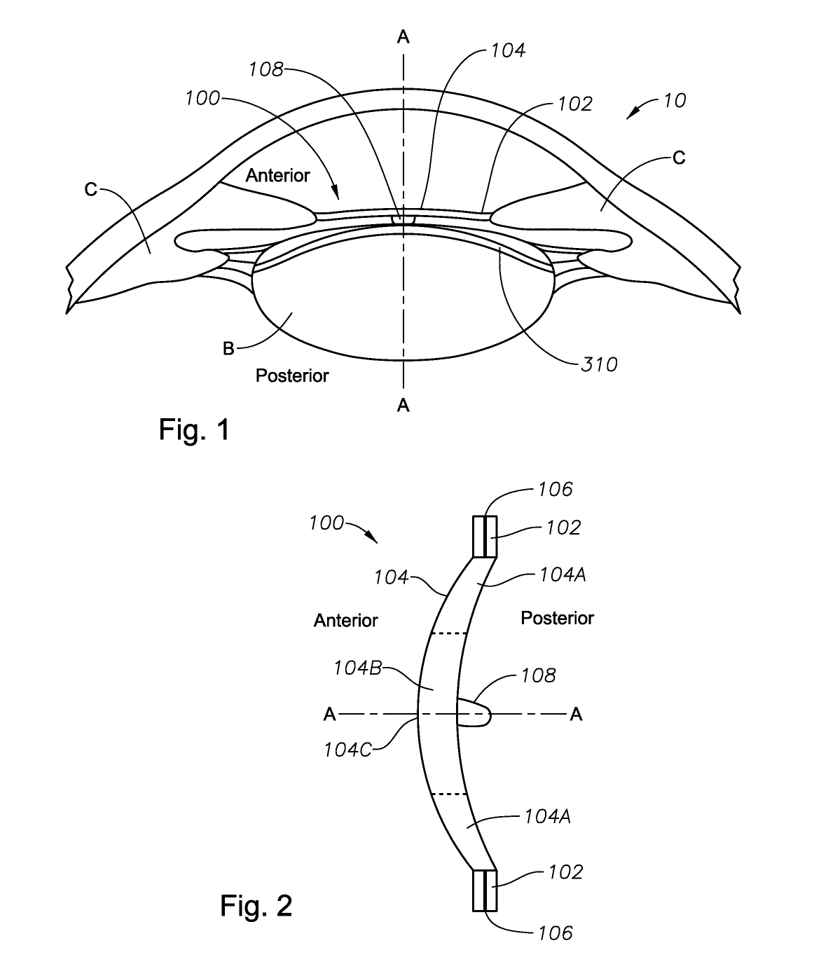

FIGS. 1-4 illustrate an accommodating lens assembly 100, according to various implementations of the invention. As illustrated in FIG. 2, lens assembly 100 includes a haptic system 102, a lens body 104, and a wing 108. Lens assembly 100 is illustrated with respect to an optical axis A-A that generally runs through the center of lens body 104. As shown in FIG. 1, the lens assembly 100 generally will be implanted within a patient's eye 10 in a position within or in front of the capsular bag B. For example, the lens assembly 100 can be implanted within the anterior chamber A of the patient's eye immediately in front of and/or engaging a forward portion of the capsular bag, with the haptics extending radially outwardly toward opposite portions of the ciliary body C of the patient's eye.

According to various implementations of the invention, lens body 104 may be formed from soft, flexible optic lens materials such as hydrophyllic and/or hydrophobic acrylic, silicone, and/or hydrogel materials. For example, lens body 104 may be formed from Acrysof® acrylic material manufactured by Alcon Laboratories.

In some implementations of the invention, lens body 104 may include an outer portion 104A that radially surrounds an inner portion 104B. In some implementations of the invention, as illustrated, lens body 104 has a generally curved configuration, where inner portion 104B includes an apex of curvature 104C at an anterior side (i.e., side away from an eye when implanted into the eye) and generally coincident with optical axis A-A and thus can provide the lens body with a base optic power.

Lens body 104 generally can be manufactured having a shape in a disaccommodated configuration. Therefore, when a deforming force is applied to lens body 104 during accommodation of the patient's eye in which the IOL is implanted., inner portion 104B may bulge or otherwise be urged in a direction generally along optical axis A-A such that when deformed, lens body 104 changes its shape (i.e., its curvature) to an accommodative configuration. In some implementations of the invention, in response to the applied deforming force when implanted into the patient's eye, inner portion 104B bulges “forward” toward the anterior chamber of the eye (i.e., away from the capsular bag).

In some implementations of the invention, the inner portion 104B of the lens body 104 may be deformed in response to the accommodative force due at least in part to a difference in rigidity between inner portion 104B and outer portion 104A. In some implementations of the invention, the difference in rigidity may be achieved by using a thinner and/or more flexible material for inner portion 104B than for outer portion 104A. In these implementations, outer portion 104A generally is less rigid than inner portion 104B because outer portion 104A is thicker than inner portion 104B.

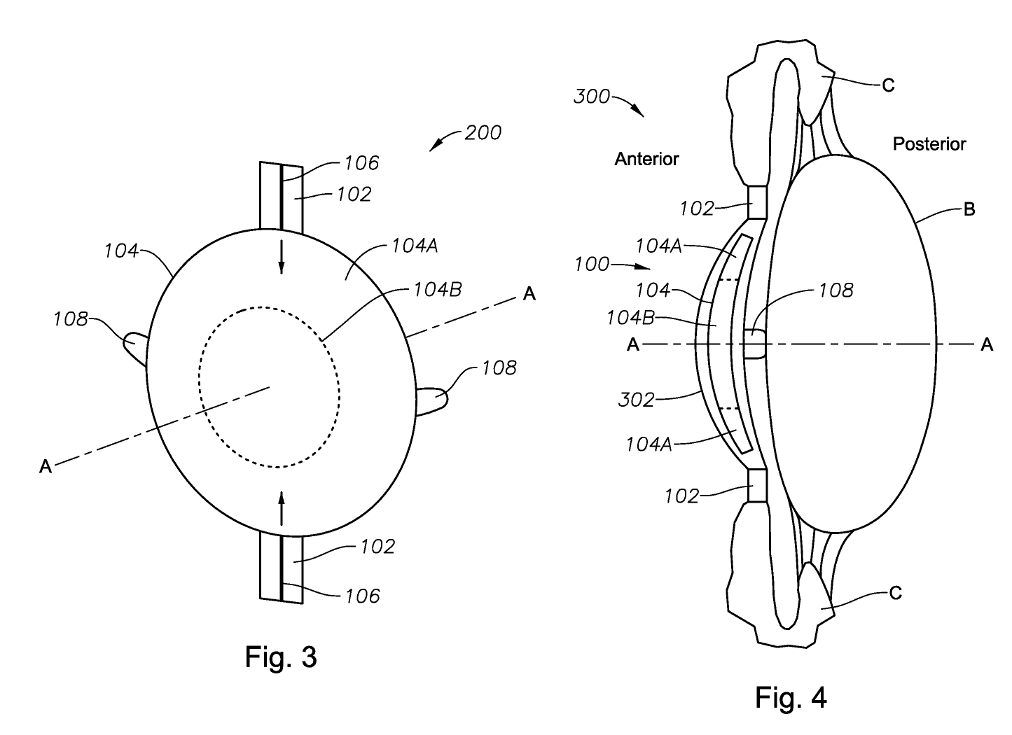

In still further implementations of the invention, the difference in rigidity also may be achieved by using different compositions of materials for inner portion 104B and outer portion 104A. For example, inner portion 104B may be formed from a material that is more flexible than a material used to form outer portion 104A. In particular, lens body 104 may be formed using one or more curing/cross-linking processes such that outer portion 104A forms into a relatively rigid structure while inner portion 104B can comprise a flexible membrane or bag that receives and contains an aqueous optic fluid material such as a liquid, gel, or soft, pliable solid materials therein. In one example, outer peripheral portion 104A may form a shell that encompasses at least a portion of the inner lens portion 104B. As an additional alternative, the difference in rigidity between the inner and outer lens portions may be achieved by forming outer and inner portions 104A, 104B using a combination of different thicknesses of material and different compositions of materials for outer and inner portions 104A, 104B. As illustrated in FIGS. 2-3, haptic system 102 includes one or more haptics 106 that extend radially outwardly from an outer peripheral portion 104A transfer force from an anatomical or other structure attached to the eye (as illustrated, for example, in FIG. 3) to lens body 104. In such implementations, haptic system 102 may engage at least a portion of a ciliary body C of an eye 10 (FIG. 1) The haptic system 102 also may be configured to engage at least a portion of the ciliary body in order to transfer force from the ciliary body during accommodation. In particular, haptics 106 may be formed to be scooped with respect to the curvature of lens body 104. As illustrated in FIG. 2, haptics 106 is substantially perpendicular to optical axis A-A to help facilitate engagement with a portion of the ciliary body. For example, haptic system 102 may transfer force during accommodation to deform lens body 104, causing lens body 104 to change power. In some implementations of the invention, haptics 106 may include a rigid structure that transmits the deforming force. In some implementations of the invention, haptics 106 can be formed with lens body 104 or can be separately formed and attached thereto by plasma bonding, adhesive attachment or other attachment means as understood in the art.

The haptic 106 typically can be integrally formed with the lens body 104 and will project radially therefrom for a length/distance adapted to extend to a position adjacent and/or in contact with the ciliary body of the patient's eye upon implantation of the lens assembly. Thus, as the ciliary body moves inwardly and forwardly during accommodation, the haptic will be engaged so as to transfer an axial compressive force to the lens body. Alternatively, the haptics can be separately formed from a similar acrylic, silicone or hydrogel optic material as the lens body, having a similar or greater rigidity to that of the outer portion 104A of the lens body to apply a consistent compressive force thereto without collapsing or otherwise inadvertently deforming, and can be attached to a peripheral side edge of the outer portion 104A by adhesive or chemical bonding, welding or other methods as known in the art.

As further illustrated in FIGS. 1-5, the, lens assembly 100 generally will also include a wing 108 that anchors lens body 104 directly or indirectly to a structure of an eye. In some implementations of the invention, wing 108 is formed with lens body 104, creating a single unitary structure. In other implementations, wing 108 can be manufactured separately from a similar rigid optic material as the outer lens body portion and/or the haptics, and can be attached to lens body 104 using various bonding or adhering processes as would be appreciated by one skilled in the art. In some implementations of the invention, wing 108 is configured to be tucked or inserted through a capsulorhexis formed in the capsular bag and will engage the capsular bag of the eye to help align the lens assembly in a desired location/placement within the eye, as illustrated in FIG. 3. Each wing 108 engages a portion of the capsular bag along the capsulorhexis such that at least a portion of lens assembly 100 is anchored in a desired location and maintained in direct contact with the capsular bag. Each wing 108 further can engage the capsular bag such that at least a minimal defined space is maintained between lens assembly 100 and the capsular bag (i.e., lens assembly 100 is not in direct contact with the capsular bag), but with the lens assembly anchored or otherwise fixed in a desired position or location in the patient's eye. By anchoring lens assembly 100 to a structure of an eye, wing 108 may stabilize lens assembly 100 during accommodation so that the deforming force imposed via haptic system 102 causes lens body to deform to change power rather than translate in a manner that hinders accommodation. In other words, wing 108 may provide stability while lens body 104 is deformed during accommodation.

FIG. 3 is a plan view of an accommodating intraocular lens assembly 200, according to various implementations of the invention. Referring to FIG. 3, reference symbols and optical axis A-A of lens assembly 100 correspond to references symbols of lens assembly 100 illustrated in FIGS. 1-4. As illustrated in FIG. 3, outer portion 104A of lens body 104 radially surrounds inner portion 104B. In some implementations of the invention, the deforming force is transmitted to lens body 104 in a radially inward matter toward optical axis A-A (as illustrated by the arrows), thereby causing lens body 104 to deform. In some implementations, the radial forces generally will be evenly distributed about lens body 104, thereby deforming lens body 104 substantially evenly in different directions.

FIG. 4 shows a side view in cross-section of an accommodating lens assembly 100 implanted into an eye 300, according to various implementations of the invention. Referring to FIG. 4, reference symbols and optical axis A-A of lens assembly 100 correspond to references symbols of lens assembly 100 illustrated in FIG. 2. Thus, the structure and functionality of these components operate in a manner similar to that described in FIG. 2. As illustrated in FIG. 4, the lens body is encapsulated by a flexible membrane 302. In some implementations of the invention, flexible membrane 302 may encapsulate lens body 104, haptic system 102, and/or wing 108. Flexible membrane 302 may be formed from various lens materials similar to the composition of lens body 104, as would be appreciated.

As indicated in FIG. 1, when implanted into a patient, lens assembly 100 generally is located in a position or location anterior to the capsular bag of the patient's eye, and can be attached, directly or indirectly, to at least a portion of an anatomical structure of the eye 300. As illustrated in FIGS. 1 and 4, for example, lens assembly is engaged by ciliary body C via haptic system 102. In some implementations of the invention, lens assembly 100 is also attached, directly or indirectly, to the capsular bag or other structure of the eye, via the wings 108 in order to anchor lens assembly 100 during accommodation. In this manner, deforming forces are directed primarily to deform lens body 104 forwardly, as described above, instead of displacing lens assembly 100 from its implanted position. As illustrated in FIG. 4, for example, one or more wings 108 anchor the lens assembly 100 to capsular bag B, engaging the capsular bag B at a capsulorhexis associated with removal of a natural lens of eye 300 such as during a cataract procedure. In these implementations, wing 108 may anchor lens assembly 100 via an incision already created during surgery.

In operation, various processes of ciliary body C may cause haptic system 102 to transmit a deforming force to lens body 104. In some implementations of the invention, ciliary body C causes haptic system 102 to transfer the deforming force in a direction that is generally inward toward and perpendicular to optical axis A-A, causing lens body 104 to bulge forward along optical axis A-A toward the anterior side and away from capsular bag B, causing lens body 104 to enter an accommodative configuration. When the accommodative deforming force is released, such as when the patient's eye returns to a disaccommodated state, lens body 104 relaxes back to its original, disaccommodative shape.

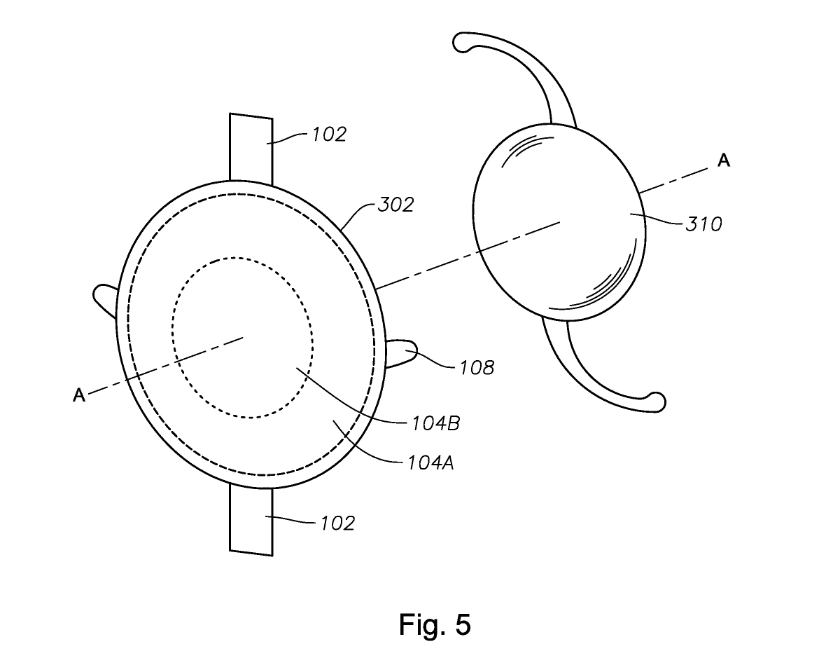

FIG. 5 is a plan view of an accommodative lens assembly 100 anterior to a capsular bag B that includes posterior lens 310, according to various implementations of the invention. Reference symbols and optical axis A-A of lens assembly 100 correspond to reference symbols of lens assembly 100 illustrated in FIG. 1. As illustrated in FIG. 5, lens assembly 100 includes both an anterior lens, at least partially formed by lens body 104, and a posterior lens 310. In some implementations, posterior lens 310 includes the natural lens of the patient's eye, and in other implementations, posterior lens 310 can be another or secondary replacement lens such as an IOL that replaces the natural lens during a cataract surgery. In these implementations, the posterior lens can have a predetermined and fixed base power, which predetermined base optical power of this posterior lens does not change during accommodation (i.e., is configured as a rigid lens that does not deform during accommodation) referenced to in FIG. 5.

FIG. 5 further illustrates an orientation and configuration of lens assembly 100 with respect to capsular bag B, whereby the lens assembly 100 is anterior to the capsular bag (i.e., the capsular bag is posterior to lens assembly 100). The plan view illustrated in FIG. 5 is for clarity and understanding only. For example, lens assembly 100 and capsular bag B are illustrated in FIG. 5 as detached from one another, although in operation, wing(s) 108 attaches lens assembly 100 to the capsular bag or other structure. In some implementations, one or more wing(s) 108 may attach lens assembly 100 to the capsular bag such that they are in contact with one another. In other implementations, one or more wing(s) 108 may attach lens assembly 100 to capsular bag B in a spaced, separated position such that they do not directly contact each other.

In operation, lens body 104 has an optic power when in a disaccommodative shape/curvature. During accommodative movement of the ciliary body of the patient's eye, a haptic element of haptic system 102 is engaged and causes lens body 104 to deform, altering the curvature of the lens body to adjust the optic power. In this manner, the anterior lens changes power during accommodation. Engagement of the wing(s) 108 with the capsular bag helps locate and secure the lens body in a desired position within the patient's eye and can further provide support to help the lens body resist buckling and maintains the anterior lens in its implanted position in the patient's eye. In some implementations, the posterior lens maintains its optic power during accommodation. In these implementations, the posterior lens may provide a base power that does not change during accommodation while the anterior lens can change optic power during accommodation.

Implementations of the invention may be described as including a particular feature, structure, or characteristic, but every aspect or implementation may not necessarily include the particular feature, structure, or characteristic. Further, when a particular feature, structure, or characteristic is described in connection with an aspect or implementation, it will be understood that such feature, structure, or characteristic may be included in connection with other implementations, whether or not explicitly described. Thus, various changes and modifications may be made to the provided description without as would be appreciated. As such, the specification and drawings should be regarded as illustrative only, and the scope of the invention to be determined solely by the appended claims.

What is claimed is:

1. An accommodative intraocular lens assembly for implantation in a patient's eye, comprising:
a lens body having a base optical power;
a haptic system coupled to the lens body, the haptic system configured to engage at least a portion of a ciliary body of the patient's eye; and
a wing coupled to the lens body, the wing configured to fit within an anterior capsulorhexis in a capsular bag anterior to either a natural lens or an artificial lens, wherein, when the wing engages the capsular bag, the lens body is anterior to the capsular bag and has a curvature corresponding to a disaccommodative state of the lens body, and
wherein movement by the ciliary body during accommodation causes the haptic system to deform the lens body, changing the curvature of the lens body to an accommodative state and adjusting the optical power of the lens body, while the wing stabilizes the lens body via the capsular bag.

2. The accommodative intraocular lens assembly of claim 1, further comprising:
a second intraocular lens that replaces a natural lens and is to be implanted within the capsular bag.

3. The accommodative intraocular lens assembly of claim 2, wherein the lens body is configured to change optical power during accommodation and the second intraocular lens is configured to maintain optical power during accommodation.

4. The accommodative intraocular lens assembly of claim 1, wherein the wing is configured to engage a capsulorhexis associated with removal of a natural lens of the eye.

5. The accommodative intraocular lens assembly of claim 1, wherein the lens body comprises an inner lens portion and an outer, peripheral portion, at least a portion of the inner lens portion bulging forward in a direction away from the capsular bag when the lens body is deformed.

6. The accommodative intraocular lens assembly of claim 5, wherein the inner lens portion comprises a less dense ophthalmic material than an ophthalmic material of the outer peripheral portion, enabling bulging of the inner lens portion outwardly from the outer peripheral portion when the lens body is deformed.

7. The accommodative intraocular lens assembly of claim 1, further comprising a flexible membrane that encapsulates at least a portion of the lens body.

8. The accommodative intraocular lens assembly of claim 1, wherein the haptic system comprises a plurality of haptics each configured to provide a deforming force at radial angles during accommodation.

9. An intraocular lens assembly for correction of vision in a patient's eye, comprising:
an anterior lens including a lens body having a base optic power when the anterior lens is in a disaccommodative state, wings projecting radially from the lens body and adapted to engage a capsular bag portion of the patient's eye and at least one haptic element extending from the lens body to a position to be engaged by a ciliary body of the patient's eye during an accommodative movement thereof; and
a posterior lens received within the capsular bag and located in a position adjacent the anterior lens and having a pre-determined optic power;
wherein during accommodative movement of the ciliary body of the patient's eye, the at least one haptic element of the anterior lens is engaged and causes a deformation of the lens body of the anterior lens so as to alter a curvature of the lens body to adjust the optic power of the anterior lens while the engagement of the wings within the capsular bag portion of the patient's eye resist buckling and maintain the anterior lens in an implanted position in the patient's eye.

10. The intraocular lens assembly for correction of vision in a patient's eye of claim 9, wherein the lens body comprises an inner lens portion and an outer, peripheral portion, at least a part of the inner lens portion adopted to bulge forwardly in a direction away from the capsular bag when the lens body is deformed.

11. The intraocular lens assembly for correction of vision in a patient's eye of claim 10, wherein the inner lens portion comprises a flexible ophthalmic material having a density lesser than an ophthalmic material of the outer peripheral portion and sufficient to enable bulging of the inner lens portion outwardly from the outer peripheral portion when the lens body is deformed.

12. The intraocular lens assembly for correction of vision in a patient's eye of claim 9, wherein the posterior lens replaces a natural lens and is to be implanted with the capsular bag; and wherein the lens body of the anterior lens is configured to change optic power during accommodation while the posterior lens is configured to maintain its optic power during accommodation.
